(19) United States
(12) Patent Application Publication
(10) Pub. No.: US 2022/0313851 A1
(43) Pub. Date: Oct. 6, 2022

(54) FLEXIBLE RADIATIVE DECONTAMINATION APPARATUS AND METHOD OF USE

(71) Applicant: LUMAEGIS, INC., Austin, TX (US)

(72) Inventors: Sudhir SUBRAMANYA, Austin, TX (US); John S. MORREALE, Austin, TX (US); Werner K. GOETZ, Austin, TX (US); Matthijs Hendrik KEUPER, Geldrop (NL)

(73) Assignee: LUMAEGIS, INC., Austin, TX (US)

(21) Appl. No.: 17/845,128

(22) Filed: Jun. 21, 2022

Related U.S. Application Data
(63) Continuation of application No. PCT/US2021/048696, filed on Sep. 1, 2021.
(60) Provisional application No. 63/073,179, filed on Sep. 1, 2020.

(51) Int. Cl. A61L 2/10; A61L 2/24; A61L 2/08

(52) U.S. Cl. CPC A61L 2/10; A61L 2/24; A61L 2/085; A61L 2202/11; A61L 2202/14; A61L 2202/15

(57) ABSTRACT

A decontamination apparatus for disinfecting a surface can include a flexible textile, an array of LEDs, and a flexible cover layer. The flexible textile can have a first side facing a first direction and a second side facing a second direction opposite the first direction. The array of LEDs can be configured to output radiation in at least two separate wavelength ranges corresponding to an ultraviolet radiation range and an infrared radiation range. The flexible cover layer can cover the array of LEDs and be transparent to at least the ultraviolet radiation range. The flexible cover layer can comprise a plurality of projections configured to maintain a consistent distance between the array of LEDs and a surface to be disinfected. The flexible textile, the array of LEDs, and the flexible cover layer can be coupled together to form a flexible blanket that conforms to a contour of the surface.

### CROSS-REFERENCE TO RELATED APPLICATION(S)

This application is a continuation of International Application No. PCT/US2021/048696, filed on Sep. 1, 2021, which claims priority to U.S. Provisional Application No. 63/073,179, filed on Sep. 1, 2020. The disclosures of each of the above applications are incorporated herein by reference in their entirety.

### FIELD

The present disclosure relates to an apparatus for decontaminating surfaces, and, more particularly, to a flexible blanket that utilizes light emitting diodes to decontaminate surfaces.

### BACKGROUND

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Medical facilities and equipment are subjected to decontamination/sterilization processes to eliminate bacteria, viruses, and other germs in order to create a safe environment for patients and medical professionals. The COVID-19 global pandemic has increased the demand for such decontamination/sterilization processes, and has extended their use outside of the medical and other facilities that traditionally engaged in such processes. Current decontamination methods use liquid disinfectants, which are hazardous and require a trained applier to cover all surfaces properly. Even when applied by a trained individual, however, it may be difficult to ensure the proper use of such liquid disinfectants, which can lead to surfaces being left untreated. For simple, flat surfaces (such as a table top), the use of liquid disinfectants may not pose a difficult challenge, but for contoured or complex surfaces, such as hand rails, keypads, toilet seats, etc., such liquid disinfectants may be inadequate. Furthermore, the use of consumables (such as a liquid disinfectant) require facilities to continually acquire new consumables to ensure a sufficient supply, which also must be stored at the facility.

Accordingly, there remains a need for an improved decontamination apparatus that addresses the above described and other disadvantages.

### SUMMARY

According to certain aspects of the present disclosure, a decontamination apparatus for disinfecting a surface is disclosed. The decontamination apparatus can comprise a flexible substrate, an array of LEDs, a processor, and a flexible cover layer. The flexible substrate has a first side facing a first direction and a second side facing a second direction opposite the first direction. The array of LEDs can be arranged on the first side of the flexible substrate and be configured to output radiation in at least two separate wavelength ranges. The at least two separate wavelength ranges can correspond to an ultraviolet radiation range and an infrared radiation range. The processor can be operationally coupled to the array of LEDs and configured to control the output of radiation therefrom. The flexible cover layer can be arranged to encase the array of LEDs on the first side and be transparent to the radiation in the at least two separate wavelength ranges. The flexible substrate can also include a reflective layer arranged to reflect the radiation output from the array of LEDs such that the radiation is output in the first direction and is inhibited from being output in the second direction, as well as a heat conductive layer to conduct heat generated by the array of LEDs.

In some aspects, the flexible substrate can include a textile layer, and the array of LEDs can be arranged between the textile layer and the reflective layer. The reflective layer can also define a plurality of apertures, where each of the LEDs in the array of LEDs can be arranged within one of the plurality of apertures.

In some aspects, the flexible cover layer can comprise a plurality of projections extending from the first side configured to maintain a consistent distance between the array of LEDs and a surface to be disinfected. Each of the plurality of projections comprises a cavity, e.g., a hollow cavity or otherwise.

In some aspects, the decontamination apparatus can further comprise a temperature sensor arranged on the flexible substrate and operationally coupled to the processor. The processor can control the output of radiation based on a temperature signal received from the temperature sensor.

In some aspects, the decontamination apparatus can further comprise a proximity sensor operationally coupled to the processor. The processor can deactivate the array of LEDs to stop the output of radiation when the proximity sensor detects a user within a proximity of the proximity sensor.

In some aspects, the decontamination apparatus can further comprise an image sensor operationally coupled to the processor. The processor can determine a type of a surface to be disinfected based on an image signal received from the image sensor. Alternatively or additionally, the processor can: (i) determine a type of pathogen on a surface to be disinfected based on an image signal received from the image sensor, and (ii) control the output of radiation based on the determined type of pathogen.

In some aspects, the processor can control the output of radiation by sequentially outputting radiation in the at least two separate wavelength ranges. In additional or alternative aspects, the processor can control the output of radiation by simultaneously outputting radiation in the at least two separate wavelength ranges.

In some aspects, the at least two separate wavelength ranges can correspond to a 700-1000 nanometer wavelength range and a 200-280 nanometer wavelength range.

According to certain other aspects of the present disclosure, an alternative decontamination apparatus for disinfecting a surface can include a flexible textile, an array of LEDs, and a flexible cover layer. The flexible textile can have a first side facing a first direction and a second side facing a second direction opposite the first direction. The array of LEDs can be configured to output radiation in at least two separate wavelength ranges. The at least two separate wavelength ranges can correspond to an ultraviolet radiation range and an infrared radiation range. The array of LEDs can be coupled to the textile such that the radiation is output in the first direction and is inhibited from being output in the second direction. The flexible cover layer can cover the array of LEDs and be transparent to at least the radiation in the ultraviolet radiation range. The flexible cover layer can comprise a plurality of projections configured to maintain a consistent distance between the array of LEDs and a surface to be disinfected. The flexible textile, the array of LEDs, and the flexible cover layer can be coupled together to form a flexible blanket that conforms to a contour of the surface to be disinfected.

The decontamination apparatus of claim 14, wherein the flexible textile comprises two textile layers, and wherein the array of LEDs is arranged between the two textile layers.

In some aspects, a first textile layer of the two textile layers can define a plurality of apertures. Each of the LEDs in the array of LEDs can be arranged within one of the plurality of apertures.

In some aspects, the flexible cover layer can comprise a plurality of projections extending from the first side that are configured to maintain a consistent distance between the array of LEDs and a surface to be disinfected. In some aspects, each of the plurality of projections can comprise a cavity.

In some aspects, the flexible blanket can further comprise a heat conductive layer to conduct heat generated by the array of LEDs.

In some aspects, the decontamination apparatus can further comprise a processor operationally coupled to the array of LEDs and configured to control the output of radiation therefrom. The decontamination apparatus can further comprise a temperature sensor and a proximity sensor operationally coupled to the processor, wherein the processor: (i) controls the output of radiation based on a temperature signal received from the temperature sensor, and (ii) deactivates the array of LEDs to stop the output of radiation when the proximity sensor detects a user within a proximity of the proximity sensor.

In some aspects, the decontamination apparatus can further comprise an image sensor operationally coupled to the processor, wherein the processor: (i) determines a type of pathogen on a surface to be disinfected based on an image signal received from the image sensor, and (ii) controls the output of radiation based on the determined type of pathogen.

According to certain other aspects of the present disclosure, a method of disinfecting a surface is disclosed. The method can include positioning a decontamination apparatus proximate the surface to be disinfected, where the decontamination apparatus comprises an array of LEDs, and controlling the array of LEDs to output radiation in at least two separate wavelength ranges corresponding to an ultraviolet radiation range and an infrared radiation range.

In some aspects, controlling the array of LEDs to output radiation in at least two separate wavelength ranges can comprise controlling the array of LEDs to sequentially output the radiation.

In some aspects, controlling the array of LEDs to output radiation in at least two separate wavelength ranges can comprise controlling the array of LEDs to simultaneously output the radiation.

In some aspects, controlling the array of LEDs to output radiation in at least two separate wavelength ranges can comprise controlling the array of LEDs to: (i) output radiation in the infrared radiation range such that a temperature of the surface is at least 45 degrees Celsius at a first time, and (ii) output radiation in the ultraviolet radiation range at a second time after the first time.

In some aspects, the at least two separate wavelength ranges can correspond to a 700-1000 nanometer wavelength range and a 200-280 nanometer wavelength range

Further areas of applicability of the present disclosure will become apparent from the detailed description provided hereinafter. It should be understood that the detailed description and specific examples are intended for purposes of illustration only and are not intended to limit the scope of the disclosure.

### BRIEF DESCRIPTION OF THE DRAWINGS

The present disclosure will become more fully understood from the detailed description and the accompanying drawings, wherein:

FIG. 1 is a perspective, partially schematic view of an example decontamination apparatus according to some aspects of the present disclosure;

FIG. 2 is a partial sectional view of an example decontamination apparatus according to some aspects of the present disclosure;

FIG. 3 is partial sectional view of another example decontamination apparatus in contact with a surface to be disinfected according to some aspects of the present disclosure;

FIGS. 4A and 4B are a perspective, partially schematic view and a partial sectional view, respectively, of yet another example decontamination apparatus in contact with a surface to be disinfected according to some aspects of the present disclosure;

FIG. 5 is a partial schematic view of another example decontamination apparatus applied to decontaminate a wheelchair according to some aspects of the present disclosure;

FIG. 6 is a partial schematic view of another example decontamination apparatus applied to decontaminate a toilet according to some aspects of the present disclosure; and

FIG. 7 is a partial schematic view of another example decontamination apparatus applied to decontaminate a table according to some aspects of the present disclosure.

### DETAILED DESCRIPTION

As discussed above, a need exists for an improved decontamination apparatus that more easily and completely decontaminates, disinfects, etc. a surface and without the use of liquid disinfectants. Further, a need exists for an improved decontamination apparatus that decontaminates, disinfects, etc. a surface in a short time period and that can also be utilized by a relatively unskilled operator. While the use of UV radiation (e.g., UV-C radiation) for disinfection of pathogens on surfaces is well known, there are many disadvantages associated with existing systems. For example only, due to the effective radiation dosage decreasing as the distance from the radiation sources increases, existing systems require very high powered radiation sources to ensure sufficient disinfection occurs. Furthermore, although there are existing disinfection systems that utilize heat to perform sterilization, these systems utilize extremely high temperatures (150-170° Celsius) and require a lengthy application (an hour or longer) to be effective. These temperatures may be impractical for certain surfaces (plastics, electronics, etc.) that could be damaged or destroyed by these extreme temperatures.

To address these and other needs, the present disclosure is directed to a decontamination apparatus that includes an array of LEDs arranged on a flexible substrate to create a decontamination “blanket” that conforms to the surface of an object to be decontaminated. The decontamination apparatus utilizes both ultraviolet (“UV”) and infrared (“IR”) radiation output from the array of LEDs to decontaminate a surface. The flexibility of the decontamination apparatus permits the array of LEDs to be placed in close proximity to the entire surface to be disinfected, thereby ensuring a more consistent radiation dosage across the entire surface. Furthermore, in some embodiments, the decontamination apparatus can include a flexible cover layer encasing the array of LEDs. The flexible cover layer can be transparent to the UV and/or IR radiation. The flexible cover layer can also include a plurality of projections extending outwardly from the array of LEDs to maintain a consistent distance between the array of LEDs and the surface to be treated. In this manner, the disclosed decontamination apparatus can consume less power than systems in which the distance between the radiation source and the surface to be treated cannot be controlled. Examples of specific configurations of various decontamination apparatuses are described below. It should be appreciated that these examples are not limiting, and aspects of the described examples can be combined, excluded, etc. to form a decontamination apparatus in accordance with this disclosure.

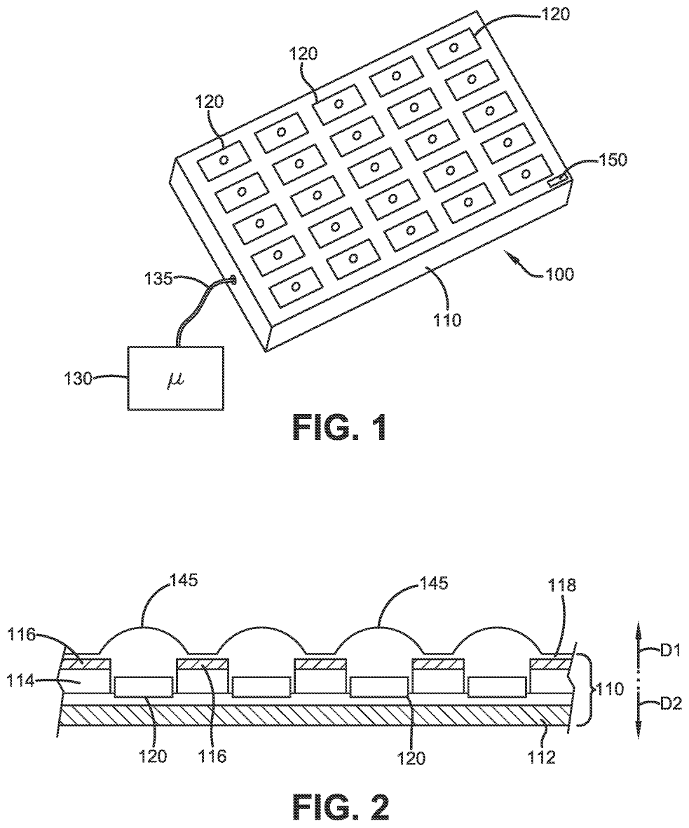

Referring now to FIGS. 1 and 2, an example decontamination apparatus 100 according to some aspects of the present disclosure is illustrated. The decontamination apparatus 100 includes a flexible substrate 110, an array of LEDs 120 arranged on the flexible substrate 110, and a flexible cover layer 140 projecting from the array of LEDs 120. The flexible substrate 110 has a first side 111 facing a first direction D1 and a second side 113 facing a second direction D2 opposite the first direction D1. In various aspects, the flexible substrate 110 comprises a flexible textile or fabric that forms flexible panel or blanket that is capable of being bent, rolled, folded, or otherwise positioned to conform to a surface(s) upon which it is arranged. The flexible substrate 110 can comprise a single layer or multiple layers coupled together, as more fully described below. In some aspects, the flexible substrate/flexible textile 110, the array of LEDs 120, and the flexible cover layer 140 are coupled to together to form a flexible blanket. As described more fully below, the flexible blanket can conform to a contour of the surface to be disinfected in order to provide a more consistent radiation dosage across the surface.

The decontamination apparatus 100 can further comprise a processor 130 that is operationally coupled to the array of LEDs 120 and configured to control the output of radiation therefrom. As described more fully below, the decontamination apparatus 100 can further include one or more sensors 150 for sensing operating characteristics during use of the decontamination apparatus 100, which can be utilized by the processor 130 to control output of radiation therefrom.

With specific reference to FIG. 2, an example of a multi-layer flexible substrate 110 is illustrated. The illustrated flexible substrate 110 of FIG. 2 includes a first textile layer 112, a heat conductive layer 114, a reflective layer 116, and a second textile layer 118. In some aspects, the first textile layer 112 comprises a single piece of a flexible textile that forms the base upon which the other layers/elements are arranged. The textile layer 112 can optionally be formed of a material having anti-microbial properties, such as linen, merino wool, hemp, polyester, polyester-vinyl composites, vinyl, or any other suitable material. Alternatively or additionally, the textile layer 112 can be coated or otherwise treated with an anti-microbial agent. Additionally or alternatively, the textile layer 112 can optionally be formed of a waterproof/water-resistant material, or be treated to be waterproof/water-resistant.

As shown in FIG. 2, the heat conductive layer 114 is arranged proximate to the textile layer 112. The heat conductive layer 114 can be formed of any material suitable for conducting heat, such as a silicone or aluminum film, a pyrolytic graphite sheet, or a spray on thermally conducting glue. As described more fully below, the heat conductive layer 114 facilitates the transfer of heat to provide a more consistent temperature across the decontamination apparatus 100. In the illustrated example, the reflective layer 116 is arranged proximate to the heat conductive layer 114. The reflective layer 116 can comprise and/or be formed of any material suitable for reflecting, guiding, or directing the radiation generated by the array of LEDS 120, e.g., an expanded polytetrafluoroethylene (“ePTFE”) fabric or a thin aluminum film. For example only, the reflective layer 116 can be arranged to reflect the radiation output from the array of LEDs 120 such that the radiation is output in the first direction D1 and is inhibited from being output in the second direction D2. In this manner, the reflective layer 116 can direct the radiation toward the surface to be disinfected while also providing protection against the radiation, which may be harmful, being output towards a user or other object.

In the example flexible substrate 110 of FIG. 2, both the heat conductive layer 114 and the reflective layer 116 define a plurality of openings or apertures 119 in which one or more LEDs of the array of LEDs 120 can be arranged. Furthermore, although the heat conductive layer 114 and the reflective layer 116 are described as separate layers, these layers can be formed of a single layer/material (such as an aluminum film) that provides both the reflective and heat conductive functions ascribed to the heat conductive layer 114 and the reflective layer 116. It should be appreciated that the description of the heat conductive layer 114 and the reflective layer 116 as being separate layers includes the implementation of a single layer/material being used for both functions.

The second textile layer 118 can be similar to the first textile layer 112. In the illustrated implementation, the second textile layer 118 is arranged proximate to the reflective layer 116 such that the array of LEDs 120 is arranged between the first textile layer 112 and the reflective layer 116, and also between the two textile layers 112, 118. Similar to the heat conductive layer 114 and the reflective layer 116, the second textile layer 118 defines a plurality of openings or apertures 119 in which one or more LEDs of the array of LEDs 120 can be arranged. In this manner, the array of LEDs 120 can output radiation, as described more fully below, through the various layers of the decontamination apparatus 100 via the apertures 119. It should be appreciated that the arrangement of the various layers of the multi-layer flexible substrate 110 of FIG. 2 is merely an example and other configurations, combinations, etc. of the various layers are within the scope of the present disclosure.

The flexible cover layer 140 can be arranged proximate to the uppermost layer of the flexible substrate 110, which in the illustrated implementation is the second textile layer 118. As described herein, the flexible cover layer 140 can project from the array of LEDs 120 and be configured to maintain a consistent distance between the array of LEDs 120 and a surface to be disinfected. The flexible cover layer 140 can take various forms. In some aspects, the flexible cover layer 140 comprises a plurality of projections 145 extending from the first side 111 of the flexible substrate 110. In the illustrated example, the flexible cover layer 140 is a single layer of material arranged to encase the array of LEDs 120. Each of the plurality of projections 145 comprises a cavity, such as a hollow cavity, arranged over at least one LED of the array of LEDs 120. The flexible cover layer 140 (including the plurality of projections 145) can be transparent to the radiation output from the array of LEDs 120. In this manner, the flexible cover layer 140 can be arranged over the array of LEDs 120 to provide protection for the LEDs, while also not inhibiting the output of radiation therefrom.

As mentioned above, the array of LEDs 120 is configured to output radiation in at least two separate wavelength ranges: an ultraviolet (“UV”) radiation range and an infrared (“IR”) radiation range. In some implementations, the at least two separate wavelength ranges corresponds to a wavelength range of 200-280 nanometers within the UV radiation range and a wavelength range of 700-1000 nanometers within the IR radiation range. As further described below, it has been determined that combining radiation from both the UV radiation range and the IR radiation range, and more specifically the 200-280 nanometer and 700-1000 nanometer ranges, provides improved disinfection results. In some aspects, the array of LEDs 120 comprise at least two different types of LEDs, a set of UV LEDs and a set of IR LEDs. Each set of different LEDs can be arranged in a separate array that, when considered together, constitutes the array of LEDs 120.

As mentioned above, the processor 130 can be operationally coupled to the array of LEDs 120 and configured to control the output of radiation therefrom. The term “processor 130” is intended in a broad sense to include a controller, microprocessor, microcontroller, and any other hardware device configured to control, provide power to, and/or otherwise manage the operation of the decontamination apparatus 100. The processor 130 can be directly coupled to the flexible substrate 110 or, as shown in the illustrations, be operably coupled via a cord 135. Further, the processor 130 can include an internal power source, e.g., a battery (not shown) and/or be configured to plug into a separate power source (battery, electrical socket, etc.).

In some aspects, the processor 130 controls the output of radiation from the array of LEDs 120 by sequentially outputting radiation in the at least two separate wavelength ranges. That is, the processor 130 will control the array of LEDs 120 to output radiation in a first wavelength range in the at least two separate wavelength ranges at a time T₁ and then, at time T₂ after time T₁, the processor 130 will control the array of LEDs 120 to output radiation in a second (different from the first) wavelength range. For example only, it may be particularly advantageous for the processor 130 to control the array of LEDs 120 to output radiation in the IR wavelength range first, and then switch to control the array of LEDs 120 to output radiation in the UV wavelength range afterwards. In other aspects, the processor 130 can control the array of LEDs 120 to output radiation by simultaneously outputting radiation in the at least two separate wavelength ranges. In yet further aspects, the processor 130 can control the array of LEDs 120 to output radiation in the at least two separate wavelength ranges simultaneously, and then sequentially, or vice versa. It should be appreciated that any manner of controlling the array of LEDs 120 to output radiation is within the scope of the present disclosure.

In some aspects, the decontamination apparatus 100 can include one or more sensors 150 for sensing operating characteristics during use of the decontamination apparatus 100. The operating characteristics can be utilized by the processor 130 to control output of radiation from the array of LEDs 120. In some aspects, the one or more sensors 150 include a temperature sensor (e.g., arranged on or near the flexible substrate 110) that is operationally coupled to the processor 130. The processor 130 can control the output of radiation from the LEDs 120 based on a temperature signal received from the temperature sensor 150. The temperature signal can be indicative of one or measures of the temperature sensed by the temperature sensor.

Alternatively or additionally, the one or more sensors 150 can include a proximity sensor operationally coupled to the processor 130. The proximity sensor can output a proximity signal when it detects a user within a proximity of the proximity sensor. The proximity sensor can be any form of sensor capable of detecting the presence of a user or other object that enters the proximity of the decontamination apparatus 100, including but not limited to an IR sensor, motion sensor, and an image sensor. The processor 130 can operate to deactivate the array of LEDs 120 to stop the output of radiation when the proximity sensor detects a user within a proximity of the proximity sensor. In this manner, the decontamination apparatus 100 can act to protect a user from receiving a radiation dosage of UV light, which be harmful.

In further implementations, the one or more sensors 150 can include an image sensor operationally coupled to the processor 130. The image sensor can output an image signal (e.g., a digital image, IR signature, a measure of reflectivity, and/or other signals) that are indicative of, or correspond to, a set of optical characteristics of a surface to be disinfected. The image sensor can be any form of sensor capable of capturing an image or information related thereto, including but not limited to an IR sensor, a reflectometer, and an image sensor. The processor 130 can utilize the image signal to determine a type (or properties) of the surface to be disinfected. For example only, the image signal can correspond to a measure of the reflectivity of the surface, the color of the surface, or other measure of the absorption characteristics of the radiation for the surface. In some aspects, the processor 130 can utilize an artificial intelligence, neural network, or other form of machine learning algorithm to determine the type of the surface to be disinfected. In this manner, the processor 130 can control the output of radiation from the array of LEDs 120 to match the type/characteristics of the surface to be disinfected to ensure an effective and efficient decontamination process. In some aspects, the image signal output by the image sensor can be indicative of, or correspond to, a set of optical characteristics of a pathogen present on the surface to be disinfected. For example only, the image signal can be analyzed by the processor 130 (e.g., using a spectroscopy process) to identify an image signature corresponding to the pathogen(s) present on a surface. The image signature can be compared to a set of image signatures of known pathogens to determine a match. In this manner, the processor 130 can retrieve a stored decontamination procedure or setting(s) corresponding to the matched pathogen. The processor 130 can control the decontamination apparatus 100 (e.g., the array of LEDs 120) to output radiation according to the determined decontamination procedure/setting(s) corresponding to the pathogen determined to be present on the surface.

In some aspects, the one or more sensors 150 can include a location sensor operationally coupled to the processor 130. The location sensor can output a location signal corresponding to a location of the decontamination apparatus 100. The location sensor can be any form of sensor capable of detecting the location of the decontamination apparatus 100, including but not limited to a GPS sensor. The processor 130 can control the output of radiation from the array of LEDs 120 based on the location signal. For example only, the location of the decontamination apparatus 100 can be used by the processor 130 to decide what the appropriate radiation dosage for the location. Alternatively or additionally, the location signal can be used to store location data related to the location and operation of the decontamination apparatus, which can be used for auditing purposes and/or as part of a learning algorithm to determine an appropriate radiation dosage for similar surfaces to be decontaminated. In this manner, the decontamination apparatus 100 can act to protect a user from receiving a radiation dosage of UV light, which be harmful.

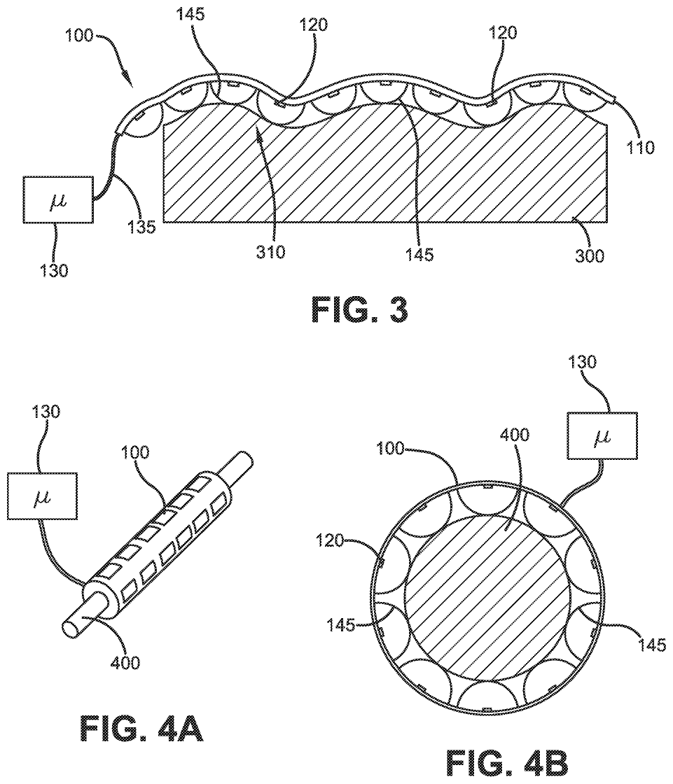

As mentioned above, the decontamination apparatus 100 can be configured to conform to a contour of a surface to be disinfected. In the illustrated example shown in FIG. 3, a partial sectional view of the decontamination apparatus 100 is shown in contact with a surface 300 to be disinfected. The decontamination apparatus 100 (with flexible substrate 110 and the array of LEDs 120) flexes and contours to the various valleys 310 and peaks 320 of the surface 300. The projections 145 of the decontamination apparatus 100 are shown in contact with the surface 300, which results in a consistent distance between the array of LEDs 120 and the surface 300 to be disinfected.

With additional reference to FIGS. 4A and 4B, a decontamination apparatus 100 is shown in contact with a cylindrical handrail 400 to be disinfected. In this example, the decontamination apparatus 100 is shown as being rolled around the cylindrical handrail 400 such that the array of LEDs 120 output radiation towards the surface to be disinfected. The decontamination apparatus 100 can include hook-and-loops connectors, snaps, buttons, or other fastening elements such that the decontamination apparatus 100 maintains a rolled configuration.

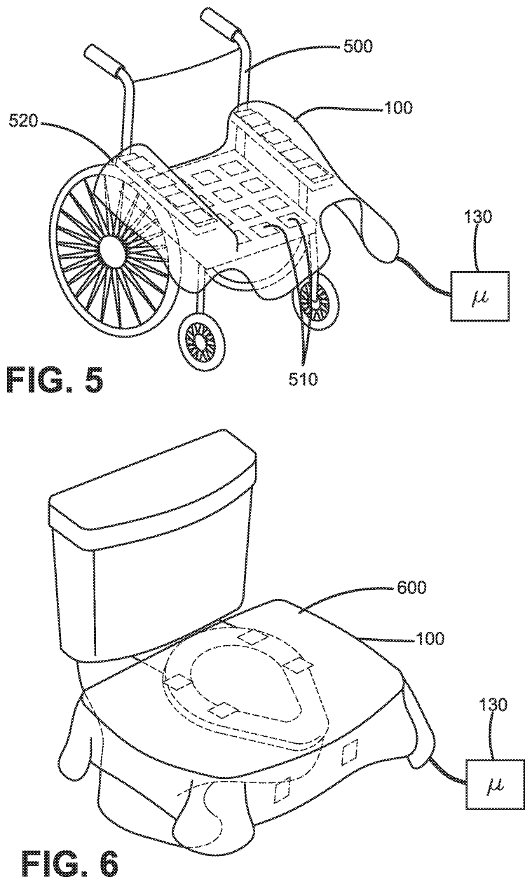

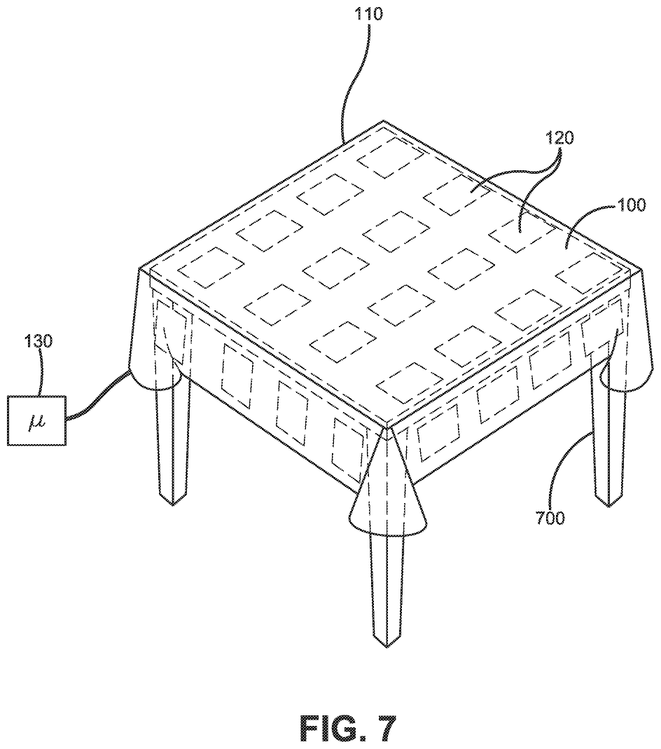

Referring now to FIG. 5, a decontamination apparatus 100 is shown in contact with a wheelchair 500 to be disinfected. In this example, the decontamination apparatus 100 is shown conforming to a seat portion 510 as well as an armrest portion 520 of the wheelchair 500. In this manner, the decontamination apparatus 100 can provide a consistent radiation dosage to the surfaces of the wheelchair during a single decontamination process. The decontamination apparatus 100 can also be used to decontaminate/disinfect a toilet 600 (FIG. 6) or a table 700 (FIG. 7) in a similar way. As shown in FIGS. 6 and 7, due to the flexible, blanket-like construction of the flexible substrate 110, the decontamination apparatus 100 will fold over surfaces to ensure a consistent radiation dosage on even the most complex, heavily contoured objects/surfaces.

The decontamination apparatus 100 of the present disclosure can be utilized to provide a safe, cost effective, and relatively quick decontamination process for objects/surfaces. Due to the specific construction and flexibility of the decontamination apparatus 100, the array of LEDs 120 can be brought into contact with (or at least closer proximity to) surfaces to be disinfected. In this manner, the array of LEDs 120 can consume less power while ensuring sufficient decontamination process than would be necessary at greater distances. Accordingly, the decontamination apparatus 100 can, in some aspects, be powered by a relatively small battery (not shown) that can provide multiple applications on a single charge.

Additionally, due to the combination resulting from the use of the at least two separate wavelength ranges, it has been observed that the decontamination apparatus 100 can provide sanitization (99.9% reduction in pathogens), decontamination/disinfection (99.99% reduction in pathogens), and/or sterilization (99.9999% reduction in pathogens) in a much shorter time duration than would be expected. For example only, it has been observed that the decontamination apparatus 100 can provide sterilization (99.9999% reduction in pathogens) in approximately thirty to sixty seconds for some surfaces by first outputting radiation in the IR wavelength range, e.g., to raise and maintain the temperature of the surface to be between 45-60° Celsius, and then outputting radiation in the UV wavelength range. The combination of the energy efficiency and speed at which disinfection can be performed, resulting from the specific construction of the decontamination apparatus 100, provides the decontamination apparatus 100 with increased utility over existing decontamination systems.

The present disclosure should not be limited to the specific example implementations of the decontamination apparatus 100 described above. It should be appreciated that feature(s) from one of the examples above could be combined with (or replace) feature(s) from another one of the examples and still be within the scope of the present disclosure. For example only, the various layers (flexible substrate 110, textile layers 112, 118, heat conductive layer 114, reflective layer 116, and flexible cover layer 140) can be combined, grouped, separated, etc. to provide a construction of a decontamination apparatus 100 more specifically suited for an intended use or surface to be disinfected. As another example, the decontamination apparatus 100 can be constructed to be of any size appropriate for the intended use. In this manner, the present disclosure describes a flexible design for a decontamination apparatus 100 that can be adapted based on the intended use and application of the decontamination apparatus 100.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known procedures, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The term “and/or” includes any and all combinations of one or more of the associated listed items. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A decontamination apparatus, comprising:
a flexible substrate having a first side facing a first direction and a second side facing a second direction opposite the first direction;
an array of LEDs arranged on the first side of the flexible substrate, the array of LEDs being configured to output radiation in at least two separate wavelength ranges, the at least two separate wavelength ranges corresponding to an ultraviolet radiation range and an infrared radiation range;
a processor operationally coupled to the array of LEDs and configured to control the output of radiation therefrom; and
a flexible cover layer arranged to encase the array of LEDs on the first side, the flexible cover layer being transparent to the radiation in the at least two separate wavelength ranges;
wherein the flexible substrate includes a reflective layer arranged to reflect the radiation output from the array of LEDs such that the radiation is output in the first direction and is inhibited from being output in the second direction, and wherein the flexible substrate includes a heat conductive layer to conduct heat generated by the array of LEDs.

2. The decontamination apparatus of claim 1, wherein the flexible substrate includes a textile layer, the array of LEDs being arranged between the textile layer and the reflective layer.

3. The decontamination apparatus of claim 2, wherein the reflective layer defines a plurality of apertures, each of the LEDs in the array of LEDs being arranged within one of the plurality of apertures.

4. The decontamination apparatus of claim 1, wherein the flexible cover layer comprises a plurality of projections extending from the first side, the plurality of projections configured to maintain a consistent distance between the array of LEDs and a surface to be disinfected.

5. The decontamination apparatus of claim 4, wherein each of the plurality of projections comprises a cavity.

6. The decontamination apparatus of claim 5, wherein the cavities comprise hollow cavities.

7. The decontamination apparatus of claim 1, further comprising a temperature sensor arranged on the flexible substrate and operationally coupled to the processor, wherein the processor controls the output of radiation based on a temperature signal received from the temperature sensor.

8. The decontamination apparatus of claim 1, further comprising a proximity sensor operationally coupled to the processor, wherein the processor deactivates the array of LEDs to stop the output of radiation when the proximity sensor detects a user within a proximity of the proximity sensor.

9. The decontamination apparatus of claim 1, further comprising an image sensor operationally coupled to the processor, wherein the processor determines a type of a surface to be disinfected based on an image signal received from the image sensor.

10. The decontamination apparatus of claim 1, further comprising an image sensor operationally coupled to the processor, wherein the processor: (i) determines a type of pathogen on a surface to be disinfected based on an image signal received from the image sensor, and (ii) controls the output of radiation based on the determined type of pathogen.

11. The decontamination apparatus of claim 1, wherein the processor controls the output of radiation by sequentially outputting radiation in the at least two separate wavelength ranges.

12. The decontamination apparatus of claim 1, wherein the processor controls the output of radiation by simultaneously outputting radiation in the at least two separate wavelength ranges.

13. The decontamination apparatus of claim 1, wherein the at least two separate wavelength ranges corresponds to a 700-1000 nanometer wavelength range and a 200-280 nanometer wavelength range.

14. A decontamination apparatus, comprising:
a flexible textile having a first side facing a first direction and a second side facing a second direction opposite the first direction;
an array of LEDs configured to output radiation in at least two separate wavelength ranges, the at least two separate wavelength ranges corresponding to an ultraviolet radiation range and an infrared radiation range, the array of LEDs coupled to the textile such that the radiation is output in the first direction and is inhibited from being output in the second direction; and
a flexible cover layer covering the array of LEDs, the flexible cover layer being transparent to at least the radiation in the ultraviolet radiation range, wherein the flexible cover layer comprises a plurality of projections configured to maintain a consistent distance between the array of LEDs and a surface to be disinfected, wherein the flexible textile, the array of LEDs, and the flexible cover layer are coupled together to form a flexible blanket that conforms to a contour of the surface to be disinfected.

15. The decontamination apparatus of claim 14, wherein the flexible textile comprises two textile layers, and wherein the array of LEDs is arranged between the two textile layers.

16. The decontamination apparatus of claim 15, wherein a first textile layer of the two textile layers defines a plurality of apertures, each of the LEDs in the array of LEDs being arranged within one of the plurality of apertures.

17. The decontamination apparatus of claim 14, wherein the flexible cover layer comprises a plurality of projections extending from the first side, the plurality of projections configured to maintain a consistent distance between the array of LEDs and a surface to be disinfected.

18. The decontamination apparatus of claim 17, wherein each of the plurality of projections comprises a cavity.

19. The decontamination apparatus of claim 14, wherein the flexible blanket further comprises a heat conductive layer to conduct heat generated by the array of LEDs.

20. The decontamination apparatus of claim 14, further comprising:
a processor operationally coupled to the array of LEDs and configured to control the output of radiation therefrom;
a temperature sensor and a proximity sensor operationally coupled to the processor, wherein the processor: (i) controls the output of radiation based on a temperature signal received from the temperature sensor, and (ii) deactivates the array of LEDs to stop the output of radiation when the proximity sensor detects a user within a proximity of the proximity sensor; and
an image sensor operationally coupled to the processor, wherein the processor: (i) determines a type of pathogen on a surface to be disinfected based on an image signal received from the image sensor, and (ii) controls the output of radiation based on the determined type of pathogen.
